(19) United States
(12) Patent Application Publication
(10) Pub. No.: US 2006/0263790 A1
(43) Pub. Date: Nov. 23, 2006

(54) Methods for improving fidelity in a nucleic acid synthesis reaction

(76) Inventor: Timothy Harris, Ocean City, NJ (US)

Correspondence Address: GOODWIN PROCTER LLP;PATENT ADMINISTRATOR, EXCHANGE PLACE, BOSTON, MA, 02109-2881, US

(21) Appl. No.: 11/133,675

(22) Filed: May 20, 2005

(51) Int. Cl. C12Q 1/68; C12P 19/34

(52) U.S. Cl. 435/6; 435/91.2; 536/25.32

(57) ABSTRACT

The invention provides methods for nucleic acid sequence determination. Generally, the invention provides methods for improving fidelity of a nucleic acid synthesis reaction.

### TECHNICAL FIELD OF THE INVENTION

The invention relates to methods for sequencing a nucleic acid, and more particularly, to methods of improving fidelity in a nucleic acid synthesis reaction. The invention improves the fidelity in a template-dependent nucleic acid synthesis reaction by exposing a target nucleic acid to a reaction mixture comprising a greater than 1 to 1 ratio of labeled deoxynucleotides to chain elongation inhibitors.

### BACKGROUND OF THE INVENTION

The ability to sequence DNA is a significant advance in the understanding of disease development and biological function. Numerous DNA sequencing techniques have been reported in literature. However, the reported sequencing techniques make it difficult or impossible to assess subtle genomic differences or changes between or within individuals. Traditional sequencing technologies also are slow because they rely on breaking a sequence into pieces, chemically manipulating the pieces, and then reassembling the pieces. As a result, emerging techniques have been devised to improve the speed and fidelity of nucleotide sequencing. For example, automated gel readers and polymerase enzymes have been introduced in order to improve sequencing efficiency and simplicity.

With the advent of a consensus human genomic sequence, the focus has shifted to individual genetic variation, and specifically, variations that can be associated with diseases of the genome. Methods, including single molecule detection methods, have provided an alternative approach designed to obtain a more direct view of molecular activity without the need to infer process or function from ensemble averaging of data. Single molecule detection creates new avenues for obtaining information on molecular structure, function and variability. While single molecule techniques have several advantages, implementation has been a problem due to failure of sequence incorporation and/or misincorporation during nucleic acid synthesis.

In a template-dependent nucleic acid synthesis reaction, the sequential addition of nucleotides is catalyzed by a nucleic acid polymerase. In practice, the fidelity of template-dependent nucleic acid sequencing depends in part on the contents of the reaction mixture. For example, minor changes in the contents of a reaction mixture can lead to unwanted results during nucleic acid synthesis. The incorporation of a nucleotide that is incorrectly paired, under standard Watson and Crick base-pairing, with a corresponding template nucleotide during primer extension may result in sequencing errors. For example, the presence of misincorporated nucleotides may result in prematurely terminated strand synthesis, reducing the number of template strands for future rounds of synthesis, and thus reducing the efficiency of sequencing.

There is, therefore, a need in the art for improved methods for reducing the frequency of misincorporation and improving fidelity in nucleic acid synthesis reactions.

### SUMMARY OF THE INVENTION

The invention improves the fidelity of template-dependent nucleic acid synthesis reactions by decreasing misincorporation rates and thereby increasing the probability that a complementary nucleotide is incorporated correctly in template-dependent synthesis. Methods of the invention comprise using unlabeled nucleotides or chain elongation inhibitors (e.g., chain-terminating nucleotides or analogs thereof) to compete with potentially misincorporating labeled nucleotides in order to minimize misincorporation. According to the invention, template-dependent nucleic acid synthesis is conducted in which template/primer duplex is exposed to a one species of labeled nucleotide and either unlabeled nucleotides, or chain-terminating analogs of one or more of the other species. The labeled nucleotide is incorporated into primer in a template-dependent manner under Watson-Crick base pairing rules. In other words, the nucleotide is incorporated into a primer at a loci at which its complement exists in the template. However, those same nucleotides are inhibited from misincorporating at other, non-complementary loci by being “out-competed” by the unlabeled or chain-terminating analogs of the complementary nucleotide at those positions. Thus, in an array of duplexes, template-dependent synthesis reactions are driven toward proper incorporation and there is a concomitant reduction in signal from misincorporated bases. Proper sequence compilation is achieved by oversampling template strands. For example, clonal populations of amplified template are used, such that some strands will be terminated or will be “unsequenceable” but because of the plurality of like templates, a consensus sequence is still obtained. The invention is especially useful to sequence short nucleotide runs, such as is the case with single nucleotide polymorphisms.

In order to limit misincorporation of non-complementary nucleotides in a template-dependent sequencing-by-synthesis reaction, the reaction mixture comprises a greater number of labeled nucleotides than unlabeled or chain elongation inhibitors. The labeled nucleotide typically will out-compete chain elongation inhibitors for complementary binding, particularly in the range of ratios provided below. Furthermore, in cases in which a chain elongation inhibitor attaches at an incorporation site intended for the complementary standard nucleotide, the chain elongation inhibitors can be cleaved and/or modified prior to subsequent addition. As an added benefit, because a complementary chain elongation inhibitor will typically out-compete a mismatched standard nucleotide for incorporation, the chain elongation inhibitor blocks misincorporation. Chain elongation inhibitors can be washed out, making their complement available for binding in subsequent nucleotide addition cycle. Similarly, in cases where labeled nucleotides are incorporated, the label can be bleached and/or cleaved prior to any subsequent synthesis.

Methods of the invention comprise conducting sequencing reactions in the presence of a reaction mixture comprising a polymerase, at least one labeled dNTP corresponding to a first nucleotide species, and at least one unlabeled dNTP that is a different species than the first nucleotide, or a chain elongation inhibitor corresponding to a nucleotide species different from that of the dNTP. For purposes of the invention, a chain elongation inhibitor is any nucleotide analog or variant that inhibits further chain elongation. For example, nucleotides comprising sterically-hindering groups are used. Also appropriate are dideoxynucleotides. In preferred embodiments of the invention, a reaction mixture comprises labeled dNTPs and ddNTPs having a ratio of dNTPs/ddNTPs greater than 1 to 1. The same ratios apply when using unlabeled dNTPs for competition at “misincorporating” sites. According to the invention, dNTPs that are complementary to an available template nucleotide will out-compete non-complementary dNTPs for template binding, resulting in reduced misincorporation.

In one aspect of the invention involving single molecule sequencing-by-synthesis, primer/target nucleic acid duplexes are bound to a surface such that one or more duplex is (are) individually optically resolvable. According to the invention, a primer/target nucleic acid (template) duplex is exposed to a polymerase, a labeled nucleotide of a first nucleotide species, and at least one unlabeled nucleotide or chain elongation inhibitor nucleotide corresponding to a different species. The duplex may be simultaneously exposed to the polymerase, labeled nucleotide and the unlabeled nucleotide or chain elongation inhibitor; or it may be first exposed to the unlabeled species or chain elongation inhibitor and then to the polymerase and labeled nucleotide. In a preferred embodiment, the duplex is simultaneously exposed to the polymerase, a single species of nucleotide and three chain elongation inhibitors, one corresponding to each of the three remaining nucleotide species. Unincorporated labeled nucleotides and/or unincorporated chain elongation inhibitors are washed away. The incorporation of the labeled nucleotide is determined, thereby revealing the identity of the complementary nucleotide at the target position (e.g., the next available base on the target just downstream of the primer). The polymerization reaction is serially repeated in the presence of labeled nucleotide that corresponds to each of the four Watson-Crick nucleotide species, and appropriate chain elongation inhibitors, until a sequence of incorporated nucleotides is compiled from which the sequence of the target nucleic acid can be determined.

Practice of the invention results in a majority of duplexes to which the added deoxynucleotide is complementary adding the appropriate (i.e., complementary) nucleotide to the primer, some duplexes adding chain-terminating nucleotides, and some duplexes to which no nucleotide is added. The proportion of chain-terminating nucleotides relative to the deoxynucleotide in any given cycle results in relatively few chain-terminating incorporations and very few or no misincorporations (due to the ability of chain-terminators, even though few in number, to outcompete any deoxynucleotides that are attempting to misincorporate).

Single molecule sequencing methods of the invention preferably comprise template/target nucleic acid duplexes attached to a surface. Individual nucleotides added to the surface comprise a detectable label—preferably a fluorescent label. Each nucleotide species can comprise a different label, or they can comprise the same label. In a preferred embodiment, the duplex is individually optically resolvable in order to facilitate single molecule discrimination. The choice of a surface for attachment of the duplex depends upon the detection method employed. Preferred surfaces for methods of the invention include surfaces comprising epoxides or a polyelectrolyte multilayer. Surfaces preferably are deposited on a substrate that is amenable to optical detection of the surface chemistry, such as glass or silica. The precise surface and substrate used in methods of the invention is, however, immaterial to the functioning of the invention described herein.

In another aspect of the invention, the target nucleic acid is a member of a clonal population of target nucleic acids. As such, methods of the invention comprise exposing a target nucleic acid, which is a member of a clonal population of target nucleic acids, to a reaction mixture comprising a primer that is complementary to a portion of the target and a polymerase capable of incorporating nucleotides to the primer in a template-dependent manner. Methods of the invention further include introducing the reaction mixture to a pre-determined ratio of labeled deoxynucleotides to unlabeled nucleotides or chain elongation inhibitors. Preferably, the ratio of labeled deoxynucleotides to chain unlabeled nucleotides or elongation inhibitor is greater than 1 to 1. More preferably, the ratio of labeled deoxynucleotides to unlabeled nucleotides or chain elongation inhibitor is from about 99 to 1 to about 999 to 1. Methods further include identifying an incorporated nucleotide, and repeating the introducing and identifying steps at least once. The steps can be repeated until a sequence of incorporated nucleotides is compiled from which the sequence of the target nucleic acid can be determined.

In this aspect, a clonal population of target nucleic acids is attached to a surface. Any surfaces for attachment of nucleic acids is useful in practice of the invention. In one embodiment, beads are used to attach one or more population of nucleic acids to be sequenced. Multiple clonal populations of nucleic acids may be placed on a single surface (e.g., a bead, a slide, a flow cell, or others as described herein). In such embodiments, detection is improved if clonal nucleic acid populations are segregated into optically-resolvable groups. As such, the identifying step may comprise obtaining a consensus signal from one or more clonal population(s). A consensus signal may be obtained according to methods and systems described herein or by other methods and systems known to those skilled in the art.

As discussed herein, the invention comprises a reaction mixture that includes labeled deoxynucleotides and unlabeled nucleotides or chain elongation inhibitors having a ratio such that the reaction mixture contains more labeled deoxynucleotides than unlabeled nucleotides or chain elongation inhibitors. As such, in one embodiment, a pre-determined ratio of labeled deoxynucleotides to unlabeled nucleotides or chain elongation inhibitors is greater than 1 to 1. In another embodiment, the pre-determined ratio of labeled deoxynucleotides to unlabeled nucleotides or chain elongation inhibitors is about 2 to 1, about 10 to 1, about 50 to 1, about 99 to 1, about 200 to 1, about 400 to 1, about 600 to 1, about 800 to 1, and about 999 to 1. In yet another embodiment, the pre-determined ratio of labeled deoxynucleotides to unlabeled nucleotides or chain elongation inhibitors is greater than about 999 to 1. Further, in still another embodiment, the pre-determined ratio is between about 1 to 1 and about 10 to 1, between about 10 to 1 and about 50 to 1, between about 50 to 1 and about 100 to 1, between about 100 to 1 and about 200 to 1, between about 200 to 1 and about 400 to 1, between about 400 to 1 and about 800 to 1, or between about 800 to 1 and about 1000 to 1.

Nucleotides useful in the invention include any nucleotide or nucleotide analog, whether naturally-occurring or synthetic. For example, preferred nucleotides include phosphate esters of deoxyadenosine, deoxycytidine, deoxyguanosine, deoxythymidine, adenosine, cytidine, guanosine, and uridine. In addition, preferred chain elongation inhibitors include nucleotide analogues that either are chain terminators which prevent further addition by the polymerase of nucleotides to the 3′ end of the primer by becoming incorporated into the primer themselves, or compete for incorporation without actually becoming incorporated. Preferably, the chain elongation inhibitors are dideoxynucleotides.

As discussed herein, where the chain elongation inhibitors are incorporated into the growing polynucleotide chain, they may be removed or inactivated after incorporation of the labeled nucleotide has been detected. As described below, 3′ to 5′ exonucleases such as, for example, exonuclease III, are able to remove dideoxynucleotides. Alternatively, the chain elongation inhibitors may be deoxynucleoside 5′-[α,β-methylene]triphosphates. These compounds are not incorporated into the primer. Other nucleotide derivatives such as, for example, deoxynucleoside diphosphates or deoxynucleoside monophosphates may be used which are also not incorporated into the chain.

Chain-terminating analogs of the invention may also be partial or temporary blockers of primer elongation. For example, nucleotide analogs may comprise 3′ blocking groups to prevent further base addition to the primer. The blocking group is removed after detection in order to allow further base addition.

Polymerases useful in the invention include any nucleic acid polymerase capable of catalyzing a template-dependent addition of a nucleotide or nucleotide analog to a primer. Depending on the characteristics of the target nucleic acid, a DNA polymerase, an RNA polymerase, a reverse transcriptase, or a mutant or altered form of any of the foregoing can be used. According to one aspect of the invention, a thermophilic polymerase is used, such as ThermoSequenase®, 9°N™, Therminator™, Taq, Tne, Tma, Pfu, Tfl, Tth, Tli, Stoffel fragment, Vent™ and Deep Vent™ DNA polymerase. Other polymerases are described below and/or are known in the art.

A detailed description of embodiments of the invention is provided below. Other embodiments of the invention are apparent upon review of the drawings and detailed description that follows.

### BRIEF DESCRIPTION OF THE DRAWINGS

FIG. 1 depicts single molecule sequencing of a target polynucleotide strand randomly anchored to a substrate or support.

FIG. 2 depicts the molecular set-up for performing single molecule sequencing using a biotin-streptavidin binding pair and Cy3 and Cy5 labels.

FIG. 3 depicts total internal reflection optical set up for single molecule sequencing.

### DETAILED DESCRIPTION OF THE INVENTION

The invention provides methods for template-dependent nucleic acid sequencing. Generally, methods comprise incorporating in a template-dependent manner at least one labeled nucleotide into a primer in the presence of a chain elongation inhibitor or unlabeled nucleotide of a different species than the labeled nucleotide. More particularly, methods include exposing a target nucleic acid to a reaction mixture comprising a primer that is complementary to a portion of the target nucleic acid, a pre-determined ratio of labeled deoxynucleotides to unlabeled nucleotides or chain elongation inhibitors and a polymerase capable of incorporating nucleotides into the primer in a template dependent manner. Methods further comprise identifying the incorporated nucleotide. Methods also further comprise repeating the exposing and identifying steps to compile the sequencing of the target nucleic acid. The chain elongation inhibitor also can be labeled. According to the invention, the ratio of the labeled nucleotide to the labeled chain elongation inhibitor is between about 99 to 1 and about 999 to 1. After incorporation, the incorporated nucleotides are identified as described herein.

The relative presence of deoxynucleotides compared to chain unlabeled nucleotides or elongation inhibitors in a reaction mixture reduces misincorporation of the deoxynucleotides, as the complementary unlabeled nucleotide or chain-elongation inhibitor outcompetes deoxynucleotides that may be susceptible for misincorporation. While applicable to bulk sequencing methods, the invention also is useful in connection with single molecule sequencing methods. Methods of the invention improve the fidelity of DNA synthesis by blocking misincorporation of a nucleotide triphosphate in target template/primer duplexes.

According to one aspect of the invention, a polymerization reaction is conducted in the presence of a polymerase, at least one labeled dNTP corresponding to a first nucleotide species, and at least one chain elongation inhibitor, such as a ddNTP, corresponding to a different nucleotide species.

Methods and compositions of the invention are well-suited for use in either single molecule sequencing techniques or where the target nucleic acid is a member of a clonal population of target nucleic acids. Surface-bound primer/target nucleic acid (template) duplexes are exposed to a polymerase, a labeled nucleotide corresponding to a first nucleotide species, and at least one chain elongation inhibitor corresponding to a different nucleotide species. The duplex may be simultaneously exposed to the polymerase, the labeled nucleotide, and the chain elongation inhibitor; or it may be first exposed to the chain elongation inhibitor and then to the polymerase and labeled nucleotide. Typically, however, the duplex is simultaneously exposed to the polymerase, the labeled nucleotide, and three chain elongation inhibitors, one corresponding to each of the three remaining nucleotide species. The duplexes are washed of unincorporated labeled nucleotides and chain elongation inhibitors, and the incorporation of labeled nucleotide is determined. The identity of the nucleotide positioned on the template opposite the incorporate nucleotide is likewise determined. The polymerization reaction is serially repeated in the presence of a labeled nucleotide that corresponds to each of the other nucleotide species in order to compile a sequence of incorporated nucleotides that is representative of the complement to the template nucleic acid.

A. Nucleotides

Various nucleotides or nucleotide analogs are useful according to the invention. For example, unbound deoxynucleotides for incorporation into a primer/target nucleic acid (template) duplex include any nucleotide or nucleotide analog, whether naturally-occurring or synthetic. For example, preferred nucleotides include phosphate esters of deoxyadenosine, deoxycytidine, deoxyguanosine, deoxythymidine, adenosine, cytidine, guanosine, and uridine. Other nucleotides useful in the invention comprise an adenine, cytosine, guanine, thymine base, an xanthine or hypoxanthine; 5-bromouracil, 2-aminopurine, deoxyinosine, or methylated cytosine, such as 5-methylcytosine, and N4-methoxydeoxycytosine. Also included are bases of polynucleotide mimetics, such as methylated nucleic acids, e.g., 2′-O-methRNA, peptide nucleic acids, modified peptide nucleic acids, locked nucleic acids and any other structural moiety that can act substantially like a nucleotide or base, for example, by exhibiting base-complementarity with one or more bases that occur in DNA or RNA and/or being capable of base-complementary incorporation, and includes chain-terminating analogs. A nucleotide belongs to a specific nucleotide species if they share base-complementarity with respect to at least one base. Nucleotide analogs particularly useful include analogs that closely resemble naturally-occurring substrates for polymerases in both chemical formula and structure.

Nucleotides for use in nucleic acid sequencing according to the invention preferably comprise a detectable label. Labeled nucleotides include any nucleotide that has been modified to include a label that is directly or indirectly detectable. Preferred labels include optically-detectable labels, including fluorescent labels or fluorophores, such as fluorescein, rhodamine, derivatized rhodamine dyes, such as TAMRA, phosphor, polymethadine dye, fluorescent phosphoramidite, Texas Red, green fluorescent protein, acridine, cyanine, cyanine 5 dye, cyanine 3 dye, 5-(2′-aminoethyl)-aminonaphthalene-1-sulfonic acid (EDANS), BODIPY, 120 ALEXA or a derivative or modification of any of the foregoing, and also include such labeling systems as hapten labeling. Accordingly, methods of the invention further provide for exposing the primer/target nucleic acid duplex to a digoxigenin, a fluorescein, an alkaline phosphatase or a peroxidase.

Fluorescent labeling moiety are particularly useful in methods of the invention include, but are not limited to, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′ 5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy 3; Cy5; Cy5.5; Cy7; IRD 700; IRD 800; La Jolta Blue; phthalo cyanine; and naphthalo cyanine.

Generally, useful chain elongation inhibitors include analogs that either are chain terminators which prevent further addition by the polymerase of nucleotides to the 3′ end of the chain by becoming incorporated into the chain themselves, or compete for incorporation without actually becoming incorporated. Since chain elongation by a polymerase requires a 3′ OH for the addition of a subsequent nucleotide, nucleotide analogs having a suitably modified 3′ end are useful as chain elongation inhibitors. The most commonly used chain elongation inhibitors are 2′3′-dideoxynucleosides or their derivatives, such as 2′3′-dideoxyribonucleoside triphosphates (ddNTPs) and 3′ O-methylribonucleoside 5′ triphosphates. Other useful nucleotide analogs have either a —H or a —OCH₂ moiety on the 3′ carbon of the pentose ring. Alternatively, the chain elongation inhibitor may be a nucleotide analog which has a 3′ OH group, but which, upon incorporation into the oligonucleotide product, still inhibit chain termination at some positions (Costas, Hanna, et al., Nucleic Acids Research 28: 1849-58 (2000); Hanna, M., Meth Enzymology 180: 383-409 (1989); Hanna, M., Nucleic Acids Research 21: 2073-79 (1993); Hanna, M. et al., Nucleic Acid Research 27: 1369-76 (1999)). Other chain elongation inhibitors useful in the invention include arabinonucleoside derivatives or 3′ O-methyl deoxyribonucleotide derivatives (see Sanger et al. (1977) Proc. Nat. Acad. Sci., USA 74:5463-5467; Axelrod, V. O., et al. (1978) N.A.R. 5:3549-3563.).

B. Polymerases

Nucleic acid polymerases generally useful in the invention include DNA polymerases, RNA polymerases, reverse transcriptases, and mutant or altered forms of any of the foregoing. DNA polymerases and their properties are described in detail in, among other places, DNA Replication 2nd edition, Kornberg and Baker, W. H. Freeman, New York, N.Y. (1991). Known conventional DNA polymerases useful in the invention include, but are not limited to, Pyrococcus furiosus (Pfu) DNA polymerase (Lundberg et al., 1991, Gene, 108: 1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent™ DNA polymerase, Cariello et al., 1991, Polynucleotides Res, 19: 4193, New England Biolabs), 9N™ DNA polymerase (New England Biolabs), Stoffel fragment, ThermoSequenase® (Amersham Pharmacia Biotech UK), Therminator™ (New England Biolabs), Thermotoga maritima (Tma) DNA polymerase (Diaz and Sabino, 1998 Braz J Med. Res, 31:1239), Thermus aquaticus (Taq) DNA polymerase (Chien et al., 1976, J. Bacteoriol, 127: 1550), DNA polymerase, Pyrococcus kodakaraensis KOD DNA polymerase (Takagi et al., 1997, Appl. Environ. Microbiol. 63:4504), JDF-3 DNA polymerase (from thermococcus sp. JDF-3, Patent application WO 0132887), Pyrococcus GB-D (PGB-D) DNA polymerase (also referred as Deep Vent™ DNA polymerase, Juncosa-Ginesta et al., 1994, Biotechniques, 16:820, New England Biolabs), UlTma DNA polymerase (from thermophile Thermotoga maritima; Diaz and Sabino, 1998 Braz J. Med. Res, 31:1239; PE Applied Biosystems), Tgo DNA polymerase (from thermococcus gorgonarius, Roche Molecular Biochemicals), E. coli DNA polymerase I (Lecomte and Doubleday, 1983, Polynucleotides Res. 11:7505), T7 DNA polymerase (Nordstrom et al., 1981, J Biol. Chem. 256:3112), and archaeal DP1I/DP2 DNA polymerase II (Cann et al., 1998, Proc Natl Acad. Sci. USA 95:14250-5).

While mesophilic polymerases are contemplated by the invention, preferred polymerases are thermophilic. Thermophilic DNA polymerases include, but are not limited to, ThermoSequenase®, 9°T™, Therminator™, Taq, Tne, Tma, Pfu, Tfl, Tth, Tli, Stoffel fragment, Vent™ and Deep Vent™ DNA polymerase, KOD DNA polymerase, Tgo, JDF-3, and mutants, variants and derivatives thereof.

C. Surface Chemistry

The surface chemistry provided by methods and devices described herein provides advantages for single molecule, as well as bulk sequencing applications. In some embodiments, a substrate for use in the invention is treated in order to create a surface chemistry that facilitates nucleic acid attachment and subsequent imaging. Exemplary surfaces are described in U.S. Patent Application Ser. No. 60/574,389, filed on May 25, 2004, the entire disclosure of which is incorporated by reference herein. Nucleotides are attached to the surface by conventional means. For example, nucleic acid templates may be attached via direct amine attachment to the surface, or via a binding pair, such as biotin/streptavidin, digoxigenin/anti-digoxigenin, and others known in the art. Surfaces for use in the invention are prepared to facilitate appropriate attachment chemistries. Thus, surfaces may be streptavidinated, biotinylated, or exposed to other chemistries that allow or facilitate nucleic acid attachment. In one preferred embodiment, epoxide surfaces are used in which the surface has integrated therein streptavidin. Surfaces may also comprise biotin or any other ligand binding pair member to facilitate suitable attachment.

Surface chemistries of the present invention facilitate anchoring nucleic acids on the substrate. For example, a surface negative layer may bear moieties that facilitate attachment of nucleic acid molecules, for example, by covalent linkage between these moieties and the nucleic acid molecule. Carboxylic acids, for example, are good targets for covalent bond formation. In some embodiments, a binding pair may be used, where a terminal layer bears one member of the pair, and the nucleic acid molecule bears the other. For example, streptavidin may be coupled to a surface layer of the substrate to facilitate anchoring using biotin-streptavidin binding pairs. Such treatment allows a high density of nucleic acid coverage with single molecule resolution as described in more detail below.

In another aspect of the invention, a substrate is coated with a polyelectrolyte multilayer. As such, methods for sequencing a target nucleic acid by synthesizing a complementary strand can include the steps of coating a surface of a substrate with a polyelectrolyte multilayer; permitting localization of a target nucleic acid on the surface of said substrate; providing a nucleotide including a labeling moiety; and allowing incorporation of the nucleotide into the complementary strand in the presence of a polymerase. Methods according to the invention further include detecting incorporation of the nucleotide into the complementary strand to determine the sequence of the target nucleic acid. The method may also be used in kits. The kits can be designed to carry out and facilitate the methods provided herein.

A further embodiment for preparing surfaces for single molecule detection comprises the covalent application on a surface (e.g., glass) of a charge layer, upon which an electrolyte layer is built. The covalent binding of the initial charge layer facilitates the ability of the overall charge layer (e.g., a PEM) to stick to the surface (i.e., the rinsability is improved). For example, an amine layer covalently attached to glass improves the ability of the surface attachment layer to adhere to glass. Thus, a PEM can be built on the amine layer that is wash-resistant compared to a non-covalently linked PEM. In one embodiment, the invention comprises the use of polydimethylsiloxane over which is flowed a solution of diacrylated polyethylene glycol (e.g., DAPEG SR610, Sartomer Corp. Exton, Pa.) and hexachloroplatinate (Aldrich) in a volumetric ration of about 200:1. The surface is then baked at about 80° C. for about 30 minutes, and the surface is rinsed with water to remove the diacrylated polyethylene glycol. A PEM, comprising alternating layers of polyethyleneimine and polyacrylate is then layered over the surface. Finally, the surface is coated with biotin followed by streptavidin in order to create binding sites for biotinylated nucleic acids.

D. Substrates

Substrates according to the invention can be two- or three-dimensional and can comprise a planar surface (e.g., a glass slide) or be arcuate (e.g., bead) or pointed. A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites.

Surfaces suitable for the nucleic acid detection are a significant issue in sequencing generally and single molecule sequencing in particular. Nucleotides arrayed on a solid surface have been utilized for drug development, DNA sequencing, medical diagnostics, nucleic acid-ligand binding studies and DNA computing. The principal advantages of using surface-bound nucleotides include ease of purification, conservation of material and reagents, reduction of interference between nucleotides and improved sample handling. Conventional surfaces for immobilization of DNA include latex beads, polystyrene, carbon electrodes, gold and oxidized silicon or glass. Those surfaces involve chemistries that are not ideal for sequencing of nucleic acids. A primary difficulty with most conventional surfaces is that they are susceptible to significant background radiation. When fluorescent detection is used in sequencing, that problem is significant.

Generally, a substrate may be of any suitable material that allows for single molecules to be individually optically resolvable, or that allow for multiple different clonal populations to be spatially segregated from each other. As such, devices and methods according to the invention can resolve one molecule or clonal populations from another. For example, the detection limit can be in the order of a micron. This implies that two molecules can be a few microns apart and be resolved, that is individually detected and/or detectably distinguished from each other.

Factors for selecting substrates include, for example, the material, porosity, size, and shape. In addition, substrates that can lower (or increase) steric hindrance of polymerase are preferred according to the invention. Other important factors to be considered in selecting appropriate substrates include size uniformity, efficiency as a synthesis support, and the substrate's optical properties, e.g., clear smooth substrates (free from defects) provide instrumentational advantages when detecting incorporation of nucleotides in single molecules or clonal populations.

E. Immobilization of Target Molecules on a Substrate

The target molecules or nucleic acids for use with the invention may be derived from any living or once living organisms, including but not limited to prokaryotes, eukaryotes, plants, animals, and viruses, as well as synthetic nucleic acids. The target nucleic acids may originate from any of a wide variety of sample types, including, but not limited to, cell nuclei (e.g., genomic DNA) and extranuclear nucleic acids, e.g., plasmids, and mitrochondrial nucleic acids. Nucleic acids can include DNA or RNA.

Target molecules, such as nucleic acids can be obtained from a patient sample. For example, an individual can provide a sample, such as blood, urine, cerebrospinal fluid, seminal fluid, saliva, breast nipple aspirate, sputum, stool and biopsy tissue for disease detection and analysis. Especially preferred are samples of luminal fluid because such samples are generally free of intact, healthy cells. However, any tissue or body fluid specimen may be used according to methods of the invention.

Many methods are available for the isolation and purification of target nucleic acids for use in the present invention. Preferably, the target molecules or nucleic acids are sufficiently free of proteins and any other interfering substances to allow target-specific primer annealing and extension. Preferred purification methods include (i) organic extraction followed by ethanol precipitation, e.g., using a phenol/chloroform organic reagent, preferably using an automated DNA extractor, e.g., a Model 341 DNA Extractor available from PE Applied Biosystems (Foster City, Calif.); (ii) solid phase adsorption methods; and (iii) salt-induced DNA precipitation methods, such methods being typically referred to as “salting-out” methods. Optimally, each of the above purification methods is preceded by an enzyme digestion step to help eliminate protein from the sample, e.g., digestion with proteinase K, or other like proteases.

Target molecules or nucleic acids may be synthesized on a substrate to form a substrate including regions coated with nucleic acids or primers, for example. In some embodiments, the substrate is uniformly comprised of nucleic acids targets or primers. That is, within each region in a substrate or array, the same nucleic acid or primer can be synthesized. A target nucleic acid can be immobilized or anchored on a substrate to prevent its release into surrounding solution or other medium. For example, a target nucleic acid can be anchored or immobilized by covalent bonding, non-covalent bonding, ionic bonding, Hydrogen bonding, van der Waals forces, hydrophobic bonding, or a combination thereof. The anchoring or immobilizing of a molecule to the substrate may utilize one or more binding-pairs, including, but not limited to, an antigen-antibody binding pair, a streptavidin-biotin binding pair, photoactivated coupling molecules, and a pair of complementary nucleic acids.

However, preferably, single molecules of target nucleic acids are attached to a substrate for sequence determination and analysis. In these embodiments, the nucleic acid can be attached to the substrate through a covalent linkage or a non-covalent linkage. When the nucleic acid is attached to the substrate through a non-covalent linkage, the nucleic acid includes one member of specific binding pair, e.g., biotin, the other member of the pair being attached to the substrate, e.g., avidin. Several methods are available for covalently linking polynucleotides to substrates, e.g., through reaction of a 5′-amino polynucleotide with an isothiocyanate-functionalized glass support. A wide range of exemplary linking moieties for attaching primers onto solid supports either covalently or non-covalently are known in the art.

Various configurations are possible according to methods and devices of the invention. In some embodiments, the target nucleic acids are immobilized to the surface prior to hybridization to the primer. In certain embodiments, the target nucleic acid is hybridized to the primers first and then immobilized on the surface. In still some embodiments, the primers are immobilized to the surface, and the target nucleic acids are attached to a substrate through hybridization with the primers. In some embodiments, the primer is hybridized to target nucleic acid prior to providing nucleotides or nucleotide analogs for the polymerization reaction. In some other embodiments, the primer is hybridized to the target nucleic acid while the nucleotides or nucleotide analogs are being provided. In still some embodiments, the polymerase is immobilized to the surface.

In one preferred embodiment, duplex comprising a template and a primer are each bionylated and bound to streptaviding on the surface. Thus, if melting occurs, reannealing is likely since both template and primer are bound to the surface in close proximity. It is known that steptavidin has four binding sites for biotin. Thus, template/primer pairs can be bound to the same streptavidin molecule on the surface. Alternatively, they can be bound to adjacent streptavidin molecules. The effect is the same in either embodiment—primer/template pairs have a higher likelihood of remaining annealed and available for base addition reactions. Methods for biotinylating nucleic acids are well known in the art.

Various methods can be used to anchor or immobilize the target nucleic acids or the primers to the surface of the substrate. The immobilization can be achieved through direct or indirect bonding to the surface. The bonding can be by covalent linkage. See, Joos et al., Analytical Biochemistry 247:96-101, 1997; Oroskar et al., Clin. Chem. 42:1547-1555, 1996; and Khandjian, Mole. Bio. Rep. 11:107-115, 1986. The bonding can also be through non-covalent linkage. For example, biotin-streptavidin (Taylor et al., J. Phys. D. Appl. Phys. 24:1443, 1991) and digoxigenin with anti-digoxigenin (Smith et al., Science 253:1122, 1992) are common tools for anchoring nucleic acids to surfaces and parallels. Alternatively, the attachment can be achieved by anchoring a hydrophobic chain into a lipidic monolayer or bilayer. Other methods for known in the art for attaching nucleic acids to supports also can be used.

When biotin-streptavidin linkage is used to anchor the nucleic acids, the nucleic acids can be biotinylated, while one surface of the substrates can be coated with streptavidin. Since streptavidin is a tetramer, it has four biotin binding sites per molecule. Thus, it can provide linkage between the surface and the nucleic acid. In order to coat a surface with streptavidin, the surface can be biotinylated first, and then one of the four binding sites of streptavidin can be used to anchor the protein to the surface, leaving the other sites free to bind the biotinylated polynucleotide (see, Taylor et al., J. Phys. D. Appl Phys. 24:1443, 1991). Such treatment leads to a high density of streptavidin on the surface of the substrate allowing a correspondingly high density of template coverage. Surface density of the nucleic acid molecules can be controlled by adjusting the concentration of the nucleic acids applied to the surface. Reagents for biotinylating a surface can be obtained, for example, from Vector Laboratories. Alternatively, biotinylation can be performed with BLCPA: EZ-Link Biotin LC-PEO-Amine (Pierce, Cat. 21347), or any other known or convenient method.

In some embodiments, labeled streptavidin (e.g., streptavidin bearing a label such as a fluorescent label) of very low concentration (e.g., in the μM, nM or pM range) is used to coat the substrate surface prior to anchoring. This can facilitate immobilization of the nucleic acid with single molecule resolution. It also can allow detecting spots on the substrate to determine where the nucleic acid molecules are attached, and to monitor subsequent nucleotide incorporation events.

While different nucleic acid molecules can be each immobilized to and sequenced in a separate substrate, multiple nucleic acids can also be analyzed on a single substrate. In the latter scenario, the templates can be bound to different locations on the substrate (e.g., at different locations on a glass slide). This can be accomplished by a variety of different methods, including hybridization of primer capture sequences to nucleic acids immobilized at different locations on the substrate. Where the target nucleic acid is a member of a clonal population of target nucleic acids, the target can be amplified on the surface according to methods known to those skilled in the art. Preferably, the multiple different clonal populations are amplified, but isolated such that the clonal populations are spatially segregated from each other

In certain embodiments, different nucleic acids also can be attached to the surface of a substrate randomly as the reading of each individual molecule may be analyzed independently from the others. Any other known methods for attaching nucleic acids and/or proteins may be used.

Conditions for hybridizing primers to polynucleotide targets are well known. The annealing reaction is performed under conditions which are stringent enough to guarantee sequence specificity, yet sufficiently permissive to allow formation of stable hybrids at an acceptable rate. The temperature and length of time required for primer annealing depend upon several factors including the base composition, length and concentration of the primer, and the nature of the solvent used, e.g., the concentration of cosolvents such as DMSO (dimethylsulfoxide), formamide, or glycerol, and counterions such as magnesium. Typically, hybridization (annealing) with synthetic polynucleotides is carried out at a temperature that is approximately 5 to 10° C. below the melting temperature of the target-primer hybrid in the annealing solvent. Typically, the annealing temperature is in the range of 55 to 75° C. and the primer concentration is approximately 0.2 μM. Under such conditions, the annealing reaction is usually complete within a few seconds.

F. Sequencing by Synthesis Methods

Primer extension can be conducted to sequence the target nucleic acid or primer using a polymerase, a labeled nucleotide (e.g., dATP, dTTP, dUTP, dCTP and/or a dGTP) or a labeled nucleotide analog, and a chain elongation inhibitor. Preferred chain elongation inhibitors include nucleotide analogues that either are chain terminators which prevent further addition by the polymerase of nucleotides to the 3′ end of the chain by becoming incorporated into the chain themselves, or compete for incorporation without actually becoming incorporated. Preferably, the chain elongation inhibitors are dideoxynucleotides. As described herein, a reaction mixture comprises labeled nucleotide and chain elongation inhibitor in a ratio greater than 1 to 1 (e.g., a ratio such that the reaction mixture contains more labeled deoxynucleotides than chain elongation inhibitors.) As described herein, the pre-determined ratio of labeled deoxynucleotides to chain elongation inhibitors can be from about 2 to 1, about 10 to 1, about 50 to 1, about 99 to 1, about 200 to 1, about 400 to 1, about 600 to 1, about 800 to 1, and about 999 to 1. In yet another embodiment, the pre-determined ratio of labeled deoxynucleotides to chain elongation inhibitors can be greater than about 999 to 1. Further, in still another embodiment, the pre-determined ratio can be from about 1 to 1 to about 10 to 1, between about 10 to 1 to about 50 to 1, between about 50 to 1 to about 100 to 1, between about 100 to 1 to about 200 to 1, between about 200 to 1 to about 400 to 1, between about 400 to 1 to about 800 to 1, or between about 800 to 1 to about 1000 to 1.

In one aspect, incorporation of a nucleotide or a nucleotide analog and their locations on the surface of a substrate can be detected with single molecule sensitivity according to the invention. As such, the nucleic acid-primer complex can be individually resolvable. In some aspects of the invention, single molecule resolution can be achieved by anchoring a target nucleic acid at a low concentration to a surface of a substrate coated to create surface chemistry that facilitates nucleic acid attachment and reduces background noise, and then imaging nucleotide incorporation, for example, with total internal reflection fluorescence microscopy. In another aspect, incorporation of a nucleotide or nucleotide analog can be detected by obtaining a consensus signal from a clonal population.

Alternating concentrations of nucleotides also can improve signal visualization and polymerization rate in sequence analysis. In this approach, after adding a given type of labeled nucleotide to an immobilized target nucleic acid-primer complex and allowing sufficient time for incorporation, free nucleotides and chain elongation inhibitors (as well as other reaction reagents in solution) can be washed from the substrate. As such, a much lower concentration of free nucleotides when detecting signals from incorporated nucleotides will result, thereby increasing single molecule resolution. In some embodiments, a washing step can be employed to further reduce free nucleotide concentration before detecting the incorporation signals.

In some applications of the present invention, the target nucleic acids are anchored or immobilized to the substrate surface with single molecule resolution. In such methods, as described herein, single molecule resolution is achieved by using very low concentration of the polynucleotide in the immobilization reaction. For example, a 10 pM concentration for a 80-mer nucleic acid allows attachment of the nucleic acid to the surface of a silica slide at single molecule resolution.

G. Detection of Incorporated Nucleotides

Any detection method may be used which is suitable for the type of label employed. Thus, exemplary detection methods include radioactive detection, optical absorbance detection, e.g., UV-Visible absorbance detection, optical emission detection, e.g., fluorescence or chemiluminescence. For example, extended primers can be detected on a substrate by scanning all or portions of each substrate simultaneously or serially, depending on the scanning method used. For fluorescence labeling, selected regions on a substrate may be serially scanned one-by-one or row-by-row using a fluorescence microscope apparatus, such as described in Fodor (1995) and Mathies et al. (1992). Hybridization patterns may also be scanned using a CCD camera (e.g., Model TE/CCD512SF, Princeton Instruments, Trenton, N.J.) with suitable optics (Ploem, 1993), such as described in Yershov et al. (1996), or may be imaged by TV monitoring (Khrapko, 1991). For radioactive signals, a phosphorimager device can be used (Johnston et al., 1990; Drmanac et al., 1992; 1993). Other commercial suppliers of imaging instruments include General Scanning Inc., (Watertown, Mass. www.genscan.com), Genix Technologies (Waterloo, Ontario, Canada; www.confocal.com), and Applied Precision Inc. Such detection methods are particularly useful to achieve simultaneous scanning of multiple tag complement regions.

As such, embodiments of the present invention provide for detection of a single nucleotide into a single target nucleic acid molecule. A number of methods are available for this purpose. Methods for visualizing single molecules within nucleic acids labeled with an intercalating dye include, for example, fluorescence microscopy. For example, the fluorescent spectrum and lifetime of a single molecule excited-state can be measured. Standard detectors such as a photomultiplier tube or avalanche photodiode can be used. Full field imaging with a two-stage image intensified COD camera also can be used. Additionally, low noise cooled CCD can also be used to detect single fluorescent molecules.

The detection system for the signal may depend upon the labeling moiety used, which can be defined by the chemistry available. For optical signals, a combination of an optical fiber or charged couple device (CCD) can be used in the detection step. In those circumstances where the substrate is itself transparent to the radiation used, it is possible to have an incident light beam pass through the substrate with the detector located opposite the substrate from the target nucleic acid. For electromagnetic labeling moieties, various forms of spectroscopy systems can be used. Various physical orientations for the detection system are available and discussion of important design parameters is provided in the art.

A number of approaches can be used to detect incorporation of fluorescently-labeled nucleotides into a single polynucleotide molecule. Optical setups include near-field scanning microscopy, far-field confocal microscopy, wide-field epi-illumination, light scattering, dark field microscopy, photoconversion, single and/or multiphoton excitation, spectral wavelength discrimination, fluorophore identification, evanescent wave illumination, and total internal reflection fluorescence (TIRF) microscopy. In general, certain methods involve detection of laser-activated fluorescence using a microscope equipped with a camera. It is sometimes referred to as a high-efficiency photon detection system. Suitable photon detection systems include, but are not limited to, photodiodes and intensified CCD cameras. For example, an intensified charge couple device (ICCD) camera can be used. The use of an ICCD camera to image individual fluorescent dye molecules in a fluid near a surface provides numerous advantages. For example, with an ICCD optical setup, it is possible to acquire a sequence of images (movies) of fluorophores.

Certain non-limiting aspects of the invention are further described below in the following examples.

### EXAMPLES

The following Examples provide exemplary methods and devices for sequencing in a template-dependent manner.

### Example 1

Sequencing

A substrate comprising a surface chemistry suitable for anchoring nucleic acids on the substrate are used. For example, a conventional glass slide that has been pre-treated to allow single molecules to be individually optically resolvable can be used to anchor nucleic acids. The target nucleic to be sequencing is obtained from a patient sample using methods known to those skilled in the art. For example, a clinician obtains a blood sample from an individual for analysis. The sample is isolated and purified by enzyme digestion prior to ethanol precipitation so that the target nucleic acid is sufficiently free of proteins and any other interfering substances to allow target-specific primer annealing and extension.

Target nucleic acids are immobilized or anchored to the glass slide by streptavidin-biotin binding pair. Target nucleic acids are attached so that the molecules are individually optically resolvable. The target nucleic acid is hybridized to a primer to form a target nucleic acid-primer complex (duplex) under conditions optimal for hybridiziation. Such conditions are well known to those skilled in the art. For example, the annealing reaction is performed under conditions which are stringent enough to guarantee sequence specificity, yet sufficiently permissive to allow formation of stable hybrids at an acceptable rate. Hybridization (annealing) is carried out at a temperature that is approximately 5 to 10° C. below the melting temperature of the target-primer hybrid in the annealing solvent. Typically, the annealing temperature is in the range of 55 to 75° C. and the primer concentration is approximately 0.2 μM. Under such conditions, the annealing reaction is usually complete within a few seconds.

Thereafter, primer extension can be conducted to sequence the target nucleic acid or primer using a polymerase, a labeled nucleotide (e.g., dATP, dTTP, dUTP, dCTP and/or a dGTP) and a chain elongation inhibitor. The following chain elongation inhibitors can be used: ddATP, ddTTP, ddCTP and ddGTP. The reaction mixture comprises labeled nucleotide and chain elongation inhibitor in a ratio greater than 1 to 1. The anchored duplexes are subjected to serial sequencing-by-synthesis reactions as described in Braslavsky et al. (Proc. Natl. Acad. Sci., 100: 3960-64 (2003), incorporated by reference herein) in the presence of a polymerase, labeled dideoxynucleotides corresponding to a specific nucleotide species, and chain elongation inhibitors corresponding to each of the three remaining nucleotide species. Detectable labels, such as Cy3 or Cy5 can be used.

The incorporation of a labeled nucleotide is determined and recorded. An optical emission detection system is used to detect the presence of Cy3 and/or Cy5 labeled nucleotides by scanning the glass slide serially one-by-one or row-by-row using a fluorescence microscope apparatus that detects hybridization patterns using a CCD camera (Model TE/CCD512SF, Princeton Instruments, Trenton, N.J.) with suitable optics.

The chain elongation inhibitors are removed and the reaction serially repeated with labeled nucleotide corresponding to each of the different nucleotide species and the appropriate chain elongation inhibitors in order to compile a sequence that is representative of the complement of the target nucleic acid.

### Example 2

Sequencing Using a PEM Surface

A fused silica microscope slide (1 mm thick, 25×75 mm size, Esco Cat. R130110) can be used to attach DNA templates. The slides can be first cleaned with the RCA method as described above and in WO 01/32930 (incorporated by reference herein.) Multilayer of polyallylamine/polyacrylic is absorbed to the slide. An EZ link connector can then be attached to the slides as follows: the slide is dried, scratched with diamond pencil, and then covered with a hybridization chamber. A mixture of 1:1:8 EDC:BLCPA:MES (50 mM EDC, 50 mM BLCPA, 10 mM MES) can be applied to each slide. Following incubation for 20 minutes, streptavidin can be diluted to 0.1 mg/ml is added to the slide. After 20 minutes of incubation, the slide can be washed with 200/xl of Tris 10 raM.

An exemplified scheme of coating a substrate with PEM for immobilizing polynucleotide is provided as follows:

Carboxylic acid groups are negatively charged at pH 7, and are a common target for covalent bond formation. Terminating the surface with carboxylic acid groups generates a surface which is both strongly negatively-charged and chemically reactive. In particular, amines can link to carboxylic acid groups to form amide bonds, a reaction catalyzed, for example, by carbodiimides. Thus, a molecule with biotin at one end, hydrophilic spacer, and an amine at the other end can be used to terminate the surface with biotin.

Streptavidin is capable of converting a biotin-terminated surface to a surface capable of capturing biotin. Streptavidin, which carries a slight negative charge, can be used then to attach the polynucleotide templates to be analyzed to the surface by using a biotinylated primer. A buffer with a high concentration of multivalent salt can be used in order to screen the repulsion of the negatively charged surface for the negatively-charged DNA.

To coat the PEM, glass cover slips can be first cleaned with high purity H20 (H20 deionized to 18.3 MOhm-cm and filtered to 0.2 #m) and a RCA Solution (6:4:1 mixture of HIGH PURITY H₂0, (30% NH₄0H), and (30% H₂0₂)). The cover slips can be then sonicated in 2% Micro 90 detergent for 20 minutes. After thoroughly rinsing with high purity H20, the cover slips can be stirred in gently boiling RCA solution for at least 1 hour, and rinsed again with high purity H20.

After cleaning, the glass cover slips can be submerged in PAII solution (Poly(allylamine) (PAII, +): 2 mg/ml in high purity H20, adjusted to pH 7.0) and agitated for at least 10 minutes. The cover slips can then be removed from PAII and washed with BP H20 by submerging in BP H20 with agitation, repeated for at least three times. The treatment can continue by agitation in a PAcr solution (Poly(acrylic acid) (PAcr, −): 2 mg/ml in HIGH PURITY H20, adjusted to pH 7.0) for at least 10 minutes and washed with HIGH PURITY H20. The treatment steps can then be repeated once.

After PEM coating, the PEM coated glass can be incubated with an EDC/BLCPA solution for 30 minutes. The EDC/BLCPA solution can be prepared by mixing equal amounts of 50 mM EDC solution (in MES buffer) and 50 mM BLCPA (in MES buffer) and diluting to 5 mM in MES buffer. The glass can then be rinsed with 10 mM Tris-NaCl and incubated with 0.1 mg/ml streptavidin solution for 1 hour. After washing with 10 mM Tris-NaCl, the glass can be incubated with a solution containing the polynucleotide template (for example, 10−7 M in Tris 100 mM MgC12) for 30 minutes. The glass can be again rinsed thoroughly with 10 mM Tris-NaCl.

For in-situ attachment, the microfluidic substrate can be bonded to the glass cover slip by HCl-assisted bonding. Essentially, the chips can be first washed with a surfactant (e.g., first with HIGH PURITY H20, then in 0.1% Tween 20, then rinsed again with HIGH PURITY H20). The washed microfluidic chips can then be put on the glass cover slips with a few microliters of dilute HCl (e.g., 1% HCl in HIGH PURITY H20), followed by baking at 37° C. for 1-2 hours. Such treatment can enhance the bond strength to glass (e.g., >20 psi pressure) without increasing nonspecific adsorption.

Following HCl treatment, PEM formation, biotinylation, and streptavidinylation, template attachment can be performed using essentially the same reagents and methods as described above for ex-situ attachment, except that the solutions can be injected through the channels by pressure instead of just being aliquoted onto the substrate surface.

Preparation of 10 pM Oligo: a 7G nucleic acid template is pre-hybridized with CyS-labeled primer in TRIS-MgC12 buffer. The treated slide can be examined for contamination with the TIR microscope. 200 #1 of the target nucleic acid/primer mixture is applied to each slide. Following incubation for 10 minutes, the slide can be washed with 200/xl ml of Tris 10 mM.

Addition of nucleotides and polymerase: Cy3-dCTP (20 nM), ddTTP (100 nM), ddATP (100 nM), and ddGTP (100 nM), is mixed in the ECOPOL buffer. 1 #1 Klenow 210S from stock solution (stored at −20° C.) is added to 200 microliters of the nucleotide mixture. 120 gl of the mixture can then be added on each slide. After incubation for 0 to 30 minutes (for different experiments), the slide can be examined with the TIR microscope. Unless otherwise noted, all reactions were performed at room temperature, while the reaction reagents can be kept at 4° C. or −20° C. The primer/nucleic acid hybridization reaction can be carried out with a thermocycler machine. This process can be repeated using Cy3-dTTP (100 nM), ddCTP (20 nM), ddATP (20 nM), and ddGTP (20 nM). This process can be again repeated with Cy3-dATP (100 nM), ddCTP (20 nM), ddTTP (20 nM), and ddGTP (20 nM), and so on. In order to limit misincorporation of non-complementary deoxynucleotides, the reaction mixture comprises a greater number of deoxynucleotides than dideoxynucleotides. The labeled deoxynucleotides out-compete the dideoxynucleotides for complementary binding. The hybridization reactions also can involve the step of washing out the didoexynucleotides after each nucleotide addition cycle, making their complement available for binding in the subsequent nucleotide addition cycle. Similarly, in cases where labeled nucleotides are incorporated, the label can be bleached and/or cleaved prior to any subsequent synthesis.

Single molecule resolution is achieved by using very low concentration of the polynucleotide template which ensures that only one template molecule is attached to a distinct spot on the slide. Single molecule attachment to a distinct also is confirmed by the observation of single bleaching pattern of the attached fluorophores. In the reaction described above, a concentration of about 10 pM of a 80-mer oligonucleotide template is used for immobilizing to the slide. The space between different DNA molecules attached to the surface slide is measured at a few micrometers.

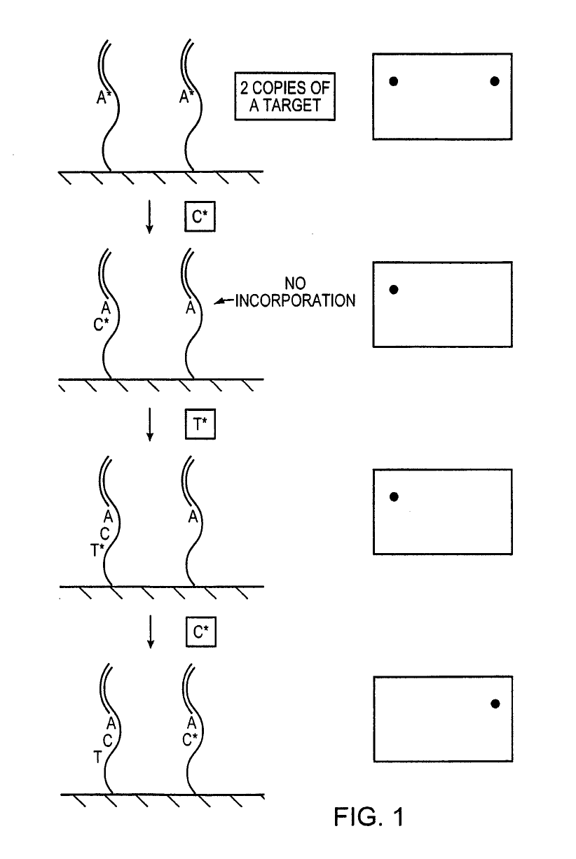

Imaging with Single Molecule Resolution: As shown in FIG. 1, incorporation of a single deoxynucleotide molecule into the complementary strand of a single target molecule can be detected and imaged according to the present invention. FIG. 1 shows two different target nucleic acids analyzed in parallel on the surface of a substrate. Incorporation of, for example, a labeled adenine deoxynucleotide (A*) into a complementary stand of one of the target nucleic acid is visualized on the surface, as indicated by the spot shown in the top view.

Later, incorporation of, for example, a labeled thymine deoxynucleotide (T*) into the complementary strand of a different target nucleic acid can be seen as a spot on a different position in the field of view, corresponding to a different location on the surface of the substrate. If labeled deoxynucleotides incorporate into both stands, for example two A*'s, two spots at corresponding positions can be detected, indicating incorporation into the complementary strands of the two individual target nucleic acids.

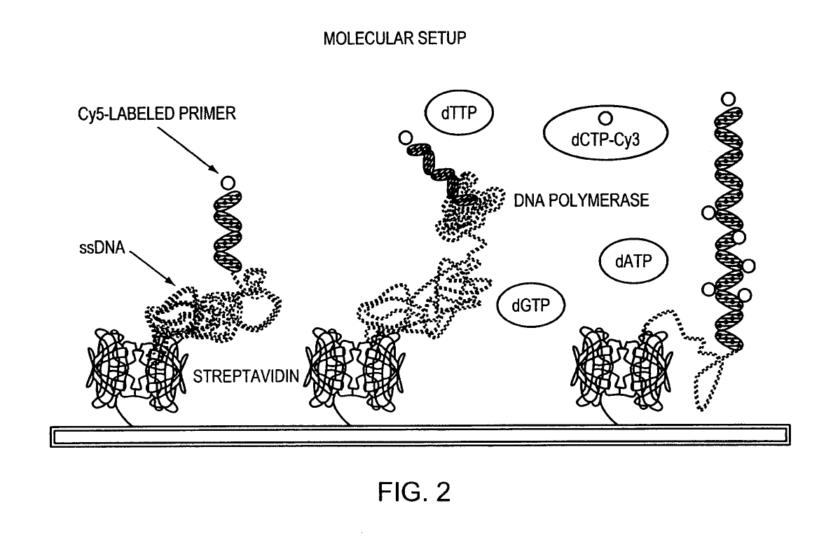

As illustrated in FIG. 2, a single stranded nucleic acid template primed with a Cy5 labeled primer sequence is immobilized at a single molecule resolution to the surface of a silica slide using a biotin-streptavidin bond. The surface is coated with polymers on which biotin (EZ link) is tethered. The nucleic acid template, with a biotin molecule attached to one of its ends, is able to attach to the streptavidin-linked surface. The slide surface is negatively charged which aids in repelling unbound nucleotides. The DNA is specifically attached to the surface by its 5′ side, meaning that the primer—which the polymerase extends—is away from the surface.

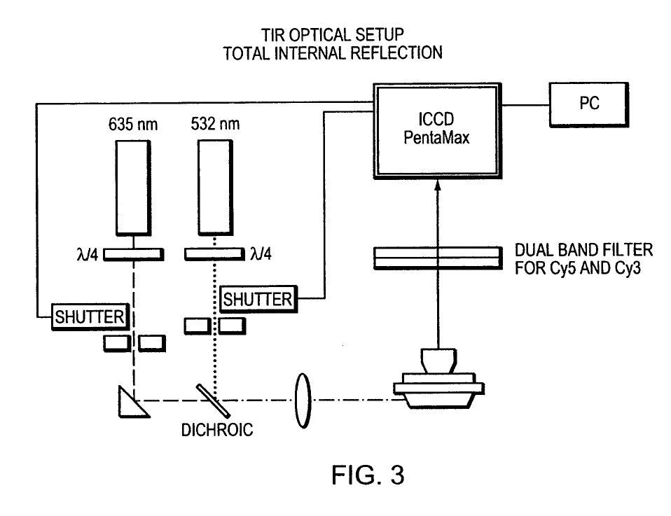

The template and incorporation of labeled nucleotides is visualized by fluorescence imaging. Location of the nucleic acid is monitored by fluorescence from the Cy5 labeled primer. Incorporation of nucleotides was detected because the nucleotides were labeled with Cy3. After incorporation, the incorporated labels were illuminated. Illumination of Cy3 was at a wavelength of 532 um. Following a typical time of a few seconds of continued illumination, the signals were bleached, typically in a single step. As shown in FIG. 3, imaging of fluorescent signals with single molecule resolution was enabled with surface illumination by total internal reflection (TIR).

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method of sequencing a nucleic acid, the method comprising the steps of:
(a) exposing a target nucleic acid to a reaction mixture comprising a primer that is complementary to a portion of said target nucleic acid, a pre-determined ratio of labeled deoxynucleotides to chain elongation inhibitors and a polymerase capable of incorporating nucleotides into said primer in a template-dependent manner;
(b) identifying an incorporated nucleotide, and
(c) repeating said exposing and identifying steps.

2. The method of claim 1, wherein said target nucleic acid is a member of a clonal population of target nucleic acids.

3. The method of claim 1, wherein said inhibitors are labeled.

4. The method of claim 2, wherein said clonal population is attached to a surface.

5. The method of claim 4, wherein said surface is a bead.

6. The method of claim 4, wherein said surface comprises multiple different clonal populations of nucleic acids.

7. The method of claim 6, wherein said multiple different clonal populations are spatially segregated from each other.

8. The method of claim 2, wherein said identifying step comprises obtaining a consensus signal from said clonal population.

9. The method of claim 1, wherein said pre-determined ratio of labeled deoxynucleotides to chain elongation inhibitors is about 2 to 1.

10. The method of claim 1, wherein said pre-determined ratio of labeled deoxynucleotides to chain elongation inhibitors is about 99 to 1.

11. The method of claim 1, wherein said labeled deoxynucleotides are attached to an optically-detectable label.

12. The method of claim 11, wherein said labeled deoxynucleotides comprise a cleavable linker to said label.

13. The method of claim 1, wherein at said deoxynucleotides and said chain elongation inhibitors contain different labels.

14. A method of sequencing a nucleic acid in a template dependent manner, the method comprising the step of:
incorporating in a template-dependent manner at least one labeled nucleotide into a primer in the presence of a labeled chain elongation inhibitor, wherein the ratio of said labeled nucleotide to said labeled chain elongation inhibitor is from about 99 to 1 to about 999 to 1.

15. The method of claim 14, further comprising the step of repeating said incorporating step sequentially.

16. The method of claim 14, further comprising the step of identifying the incorporated nucleotides.
